(19) United States
(12) Patent Application Publication
(10) Pub. No.: US 2023/0077000 A1
(43) Pub. Date: Mar. 9, 2023

(54) GAS CONCENTRATION MEASUREMENT IN GAS SEALED TROCARS

(71) Applicant: Conmed Corporation, Largo, FL (US)

(72) Inventors: Michael Koltz, JR., Aurora, CO (US); Luke Samuel Hawkins, Centennial, CO (US); Gary Tegan, Ranchero Mirage, CA (US)

(73) Assignee: Conmed Corporation, Largo, FL (US)

(21) Appl. No.: 17/467,790

(22) Filed: Sep. 7, 2021

(51) Int. Cl. A61M 13/00; A61B 5/00; A61B 10/00

(52) U.S. Cl. CPC A61M 13/006; A61B 5/4255; A61B 10/00; A61M 2205/3303; A61B 2010/0083

(57) ABSTRACT

A system for monitoring gas composition in a surgical cavity during an endoscopic surgical procedure includes an gas recirculation system including a main gas flow circuit for fluid communication with a surgical cavity. The system includes a sensor for monitoring a gas species in a gas flow from a surgical cavity of a patient. The sensor is positioned in a channel branching off from the main gas flow circuit coming from the surgical cavity.

### BACKGROUND

### 1. Field

The subject disclosure is directed to endoscopic surgery, and more particularly, to a system and method for measuring gas composition in a surgical cavity during an endoscopic or laparoscopic surgical procedure.

### 2. Description of Related Art

Unintentional and bowel perforation can occurs during laparoscopic surgery. If undetected, sepsis can develop. Due to the inherent visualization challenges associated with laparoscopic surgery, bowel perforation can escape visual detection by the surgeon. When perforation is suspected, locating and visualizing the perforation site can be difficult and time consuming.

Conventional techniques for bowel perforation detection have been considered satisfactory for their intended purpose. However, there is an ever present need for improved systems and methods for detection of a perforated bowel during an endoscopic surgical procedure, and more generally for monitoring gas species during endoscopic surgical procedures. This disclosure provides a solution for this need.

### SUMMARY

A system for monitoring gas composition in a surgical cavity during an endoscopic surgical procedure includes a gas recirculation system. The system has a main gas flow circuit for fluid communication between a surgical cavity and the gas recirculation system and a flow channel branching off from the main gas flow circuit. A sensor is operatively associated with the flow channel for monitoring a gas species in the gas flow through the main gas flow circuit. The channel branching off from the main gas flow circuit can be a dead-end channel in the gas recirculation system. The main gas flow circuit can have a cross-sectional flow area with a main diameter, wherein the sensor is spaced apart from the main gas flow circuit by a distance along the channel of ten or more main diameters distance so the sensor can detect the gas species reaching the sensor by diffusion through the channel from the main gas flow circuit.

A compressor can be operatively connected to the gas recirculation system in the main gas flow circuit to move the flow of gas to and from the surgical cavity. The channel can connect to the main gas flow circuit at a position in the main gas flow circuit downstream from the compressor. The main gas flow circuit can include an upstream portion for returning gas from the surgical cavity to the gas recirculation system, and a downstream portion for issuing gas to the surgical cavity. The compressor can separate between the upstream and downstream portions. The channel can connect to the downstream portion. A gas sealed access port can be connected to the upstream portion and to the downstream portion for connecting the gas recirculation system to the surgical cavity. The gas sealed access port, the upstream portion, the downstream portion, and the compressor can be configured to form a sealed recirculation circuit with the gas sealed access port positioned in a surgical cavity.

A controller can be operatively connected to the sensor for determining if the gas species monitored in the gas flow from the surgical cavity is within a respective desired range and taking corrective action if the gas species is outside the respective desired range. The controller can be operatively connected to a circuit of the sensor to monitor changes in electrical resistance of the sensor to determine concentration of the gas species to which the senor is exposed. A user interface can be operatively connected to the controller. The controller can be configured to issue an alert to a user through the user interface upon detection of the gas species at predetermined threshold. The controller can include a memory, wherein the controller includes machine readable instructions configured to cause the controller to write a history of gas concentration detected by the sensor for a 1 to 60 second interval into the memory for recall upon gas levels in excess of a predetermined threshold.

The sensor can include a metal oxide film and heater, configured to detect hydrogen presence in a concentration range of 100 ppm (parts per million), to 10 ppt (parts per thousand), inclusive of endpoints. The sensor can be configured so a presence of hydrogen at 10 ppt yields a drop in electrical resistance across the sensor of at or below 10 kilo-Ohms.

A method of detecting perforated bowel during a surgical procedure includes detecting presence of a gas species indicative of a perforated bowel with a sensor, wherein the gas species reaches the sensor by diffusion from a main gas flow circuit of an gas recirculation system in fluid communication with a surgical cavity. The method includes outputting an alert upon detection of the gas species at a threshold indicative of a perforated bowel.

The sensor can be located in a channel branching off from the main gas flow circuit, wherein the channel is a dead-end channel, wherein detecting includes detecting the gas species without bulk gas flow through the dead-end channel. Detecting can include sampling gas from the main gas flow circuit with the sensor, wherein sampling gas includes sampling the gas from a position in the main gas flow circuit downstream from a compressor operatively connected to the gas recirculation system in the main gas flow circuit to move the flow of gas to and from the surgical cavity. Moving the flow of gas to and from the surgical cavity can include flowing gas to and from the surgical cavity through a gas sealed access port connecting between the gas recirculation system and the surgical cavity, wherein the gas sealed access port and gas recirculation system form a sealed recirculation circuit with the gas sealed access port positioned in a surgical cavity.

A controller can be operatively connected to the sensor determining if the gas species monitored in the gas flow from the surgical cavity is within a respective desired range and to initiate output to an output device to alert a user to take corrective action if the gas species is outside the respective desired range. The controller can be operatively connected to a circuit of the sensor to monitor changes in electrical resistance of the sensor to determine concentration of the gas species to which the senor is exposed.

The method can include outputting a history of gas concentration detected by the sensor for a 1 to 60 second interval for recall upon gas levels of the gas species in excess of a predetermined threshold. The sensor can include a metal oxide film and heater, wherein detecting the gas species includes using the metal oxide film to detect hydrogen presence in a concentration range of 100 ppm (parts per million), to 10 ppt (parts per thousand), inclusive of endpoints.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 is a schematic view of an embodiment of a system constructed in accordance with the present disclosure, showing a gas recirculation system in use during a surgical procedure;

FIG. 2 is a schematic view of an embodiment of a gas delivery device; and

FIG. 3 is a schematic view of the gas delivery device of FIG. 2, showing a sensor circuit.

### DETAILED DESCRIPTION OF THE PREFERRED EMBODIMENTS

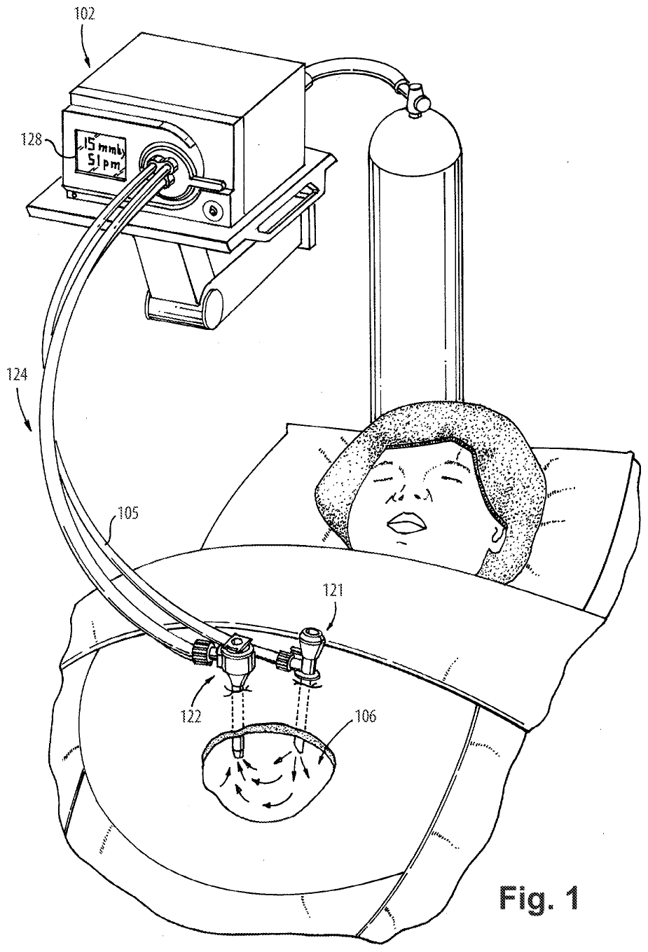

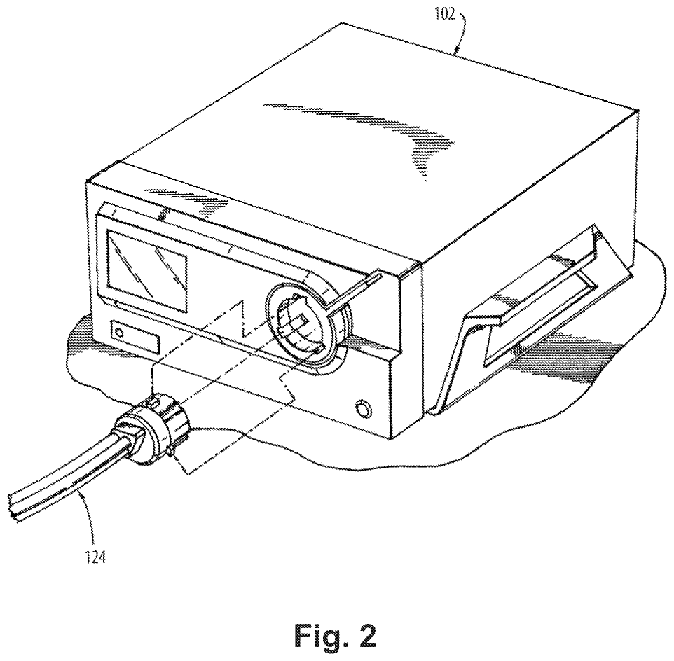

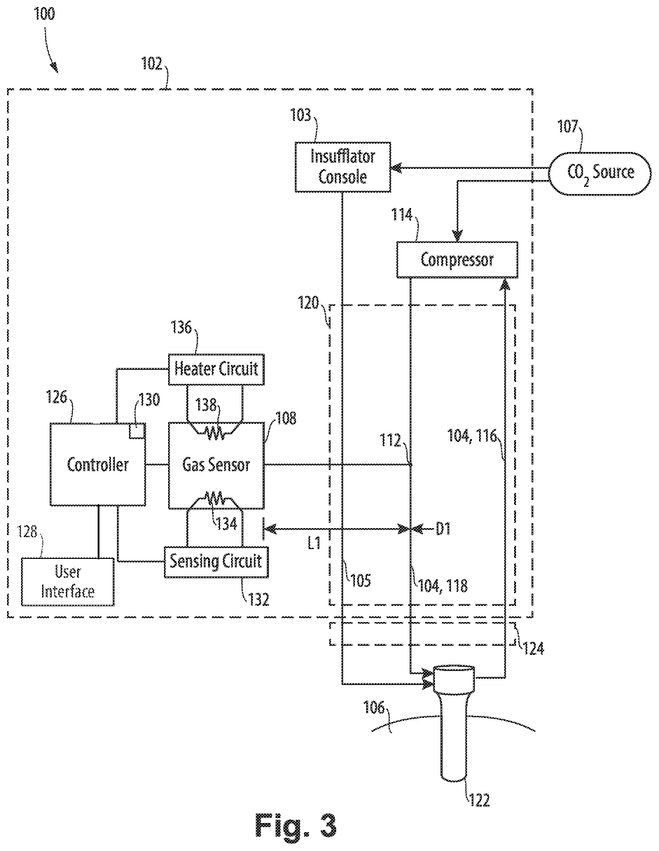

Reference will now be made to the drawings wherein like reference numerals identify similar structural features or aspects of the subject disclosure. For purposes of explanation and illustration, and not limitation, a partial view of an embodiment of a system in accordance with the disclosure is shown in FIG. 1 and is designated generally by reference character 100. Other embodiments of systems in accordance with the disclosure, or aspects thereof, are provided in FIGS. 2-3, as will be described. The systems and methods described herein can be used to monitor gas species in a surgical site during endoscopic surgical procedures, e.g., to detect perforated bowel.

As shown in FIG. 1, the system 100 includes a gas sealed access port gas recirculation system 102. The gas recirculation system 102 can be designed to cooperate with a programmable multi-modal gas recirculation system. The multi-modal gas recirculation system can be of the type described in commonly assigned U.S. Pat. Nos. 9,375,539 and 10,905,463, the disclosures of which are herein incorporated by reference in its entirety. In brief, the gas recirculation system 102 includes a multi lumen filtered tube set (e.g. tube set 124 shown in FIG. 2) including a dual lumen portion and a single lumen portion, a first gas sealed single lumen access port operatively connected to the dual lumen portion of the tube set and a second valve sealed single lumen access port operatively connected to the single lumen portion of the tube set. The dual lumen portion has a pressurized gas line and a return gas line for facilitating gas continuous recirculation relative to the jets of the gas sealed trocar, referred to herein as main gas flow circuit 104. The single lumen portion has at least a gas supply and sensing line 105 for delivering insufflation gas (e.g. from an insufflation manifold 103) to the surgical cavity 106 of the patient and for periodically sensing pressure within the surgical cavity of the patient. Additionally, the single lumen portion supplies gas at a rate in excess of the minimum needed for leak compensation to evacuate smoke from the surgical cavity. This excess flow travels back to the compressor via the return line, with entrained smoke and escaped gases from a perforated bowel, if present.

Turning now to FIG. 3, the main gas flow circuit 104 provides for fluid communication between the gas recirculation system 102 and the surgical cavity 106. The system 100 includes a sensor 108 for monitoring a gas species in a gas flow in the main gas flow circuit 104 coming from the surgical cavity 106 of the patient. The sensor 108 is positioned in a channel 110 that branches off from the main gas flow circuit 104 coming from the surgical cavity 106. The channel 110 is a dead-end channel in the gas recirculation system 102, i.e. there is an inlet 112 to the channel 110 for gas to diffuse into the channel from the main gas flow circuit 104, but there is no outlet from the channel 110. The main gas flow circuit 104 has a cross-sectional flow area with a main diameter D1. The sensor 108 is spaced apart from the main gas flow circuit 104 by a distance L1 along the channel 110 of ten or more main diameters D1 (L1≥10×D1) so the sensor 108 can detect the gas species reaching the sensor 108 by diffusion through the channel 110 from the main gas flow circuit 104. The channel 110 is depicted as non-tortuous, however more tortuous paths can be used for the channel 110 as long as the distance L1 is adjusted for the tortuosity.

A compressor 114 is operatively connected to the gas recirculation system 102 in the main gas flow circuit 104 to move the flow of gas to and from the surgical cavity 106. The compressor 144 can be any suitable compressor assembly for recirculating surgical gas through the gas sealed access port 122 by way of a gaseous sealing manifold. In certain embodiments, the compressor 114 is driven by a brushless DC (direct-current) motor, which can be advantageously controlled to adjust gas pressure and flow rates within the gas recirculation system 102. Alternatively, the compressor 114 can be driven by an AC motor, but a DC motor will be relatively smaller and lighter, and therefore more advantageous from a manufacturing standpoint.

The channel 110 connects to the main gas flow circuit 104 at a position, e.g. at the inlet 112, in the main gas flow circuit 104 downstream from the compressor 114. The main gas flow circuit 104 includes an upstream portion 116 for returning gas from the surgical cavity 106 to the gas recirculation system 102, and a downstream portion 118 for issuing gas from the compressor 114 to the surgical cavity 106. The compressor 114 separates between the upstream and downstream portions 116, 118, to drive gas flow from the surgical cavity 106, and a gas circulation valve manifold 120 regulates flow of gas to and from the surgical cavity for insufflation, stable pneumoperitoneum, smoke evacuation, and/or the like.

The gas circulation valve manifold 120 can include a first and second outlet line valve (e.g. proportional valves allowing for infinitely variable gas flow adjustment between a minimum flow state and a maximum flow state) operatively associated with the insufflation manifold 103 for controlling a flow of insufflation gas to the valve sealed access port 122. The gas circulation valve manifold 120 can include primary proportional valve is also operatively associated with insufflation manifold 103 located upstream from the first and second outlet line valves to control the flow of insufflation gas to the first and second outlet line valves.

The gas circulation valve manifold 120 can include a high pressure gas fill valve operatively associated with the compressor 114, adapted and configured to control gas delivered into the gaseous sealing manifold 110 from the source of surgical gas 107. The valve manifold can include a smoke evacuation valve that is operatively associated with the compressor 114 for dynamically controlling gas flow between the main flow path 104 and the insufflation manifold 103 under certain operating conditions.

The valve manifold can include a bypass valve positioned between an outlet side of the compressor 114 and an inlet side of the compressor 114 for controlling gas flow within the main flow path 104 under certain operating conditions. The gas circulation valve manifold 120 can also include an air ventilation valve operatively associated with an inlet side of the compressor 114 for controlling the entrainment of atmospheric air into the system 100 under certain operating conditions. For example, the air ventilation valve will permit the introduction of atmospheric air into the gaseous sealing circuit to increase the air mass (i.e., the standard volume) within the circuit.

The valve manifold can include an overpressure relief valve operatively associated with an outlet side of the compressor 114 for controlling a release of gas from the system 101 to atmosphere under certain operating conditions. The gas circulation valve manifold 120 can include a first and second blocking valve operatively associated with an inlet flow path to the main flow path 104, which can be employed during a self-test prior to a surgical procedure, as disclosed in U.S. Pat. No. 9,199,047.

The first and second blocking valves can communicate with a blocking valve pilot (e.g. solenoid valve) included within the insufflation manifold 103. The gas circulation valve manifold 120 can further include a low pressure safety valve downstream from the primary proportional valve and upstream from the first and second outlet line valves for controlling a release of gas from the system 100 to atmosphere under certain operating conditions. The valve manifold can include a ventilation exhaust valve positioned downstream from the primary proportional valve and upstream from the outlet line valves for controlling a release of gas from the system 100 to atmosphere under certain operating conditions.

The channel 110 connects to the downstream portion 118, however an embodiment where the channel 110 connects to the upstream portion 116 (upstream of the compressor 114) is also included in the scope of this disclosure. The downstream placement of the channel 110 as shown in FIG. 1 increases gas temperature proximate the sensor 108, which inherently decreases the relative humidity of the gas diffusing into the channel 110 relative to what it would be in the upstream portion 116. This is beneficial as the accuracy and life of the sensor 108 and is improved in lower relative humidity. For embodiments with placement of the channel 110 branching from the upstream portion 116, it is beneficial to compensate for the relative humidity.

A gas sealed access port 122 is connected to the upstream portion 116 and to the downstream portion 118, e.g. through tube set 124 for connecting the gas recirculation system 102 to the surgical cavity 106. The gas sealed access port 122 can be of the type disclosed in commonly assigned U.S. Pat. No. 8,795,223, which is incorporated herein by reference. The gas sealed access port 122 is adapted and configured to provide gas sealed instrument access to a body cavity, while maintaining a stable pressure within the body cavity (e.g., a stable pneumoperitoneum in the peritoneal or abdominal cavity) when used in conjunction with gas recirculation system 102 as described above. The gas sealed access port 122, the upstream portion 116, the downstream portion 118, and the compressor 114 are configured to form a continuous recirculation circuit with the gas sealed access port 122 positioned in the surgical cavity 106 (e.g. as described above). The compressor assembly 114 and its related components (which can include e.g. an intercooler/condenser, a gaseous sealing manifold and insufflation manifold 103) are all enclosed within a common housing for example as shown in FIG. 3, which includes a user interface (e.g. interface 128) and control electronics, as disclosed for example in commonly assigned U.S. Pat. No. 9,199,047, which is incorporated herein by reference.

A controller 126 is operatively connected to the sensor 108 for determining if the gas species monitored in the gas flow from the surgical cavity 106 is within a respective desired range and taking corrective action if the gas species is outside the respective desired range. Corrective action can include action by the system 100 itself (e.g. adjusting a concentration of gas(es)), or corrective action can include action by a surgeon (e.g. locating and repairing a perforation). The gas species sensor can be similar to that disclosed in commonly assigned U.S. patent application Ser. Nos. 16/000,254 and 16/000,378 both of which are incorporated herein by reference in their entirety. The user interface 128 is operatively connected to the controller 126. The controller 126 is configured to issue an alert, which can be an audio alert and/or a visual alert or written message, to a user through the user interface 128 upon detection of the gas species at, above, or near a predetermined threshold. The controller includes a memory 130, wherein the controller 126 includes machine readable instructions configured to cause the controller 126 to write a history of gas concentration detected by the sensor for a 1 to 60 second interval into the memory 130 for recall upon gas levels in excess of a predetermined threshold. The controller 126 can cause the history to be displayed on the user interface 128.

With continued reference to FIG. 3, the controller 126 is operatively connected to a sensing circuit 132 of the sensor 108 to monitor changes in electrical resistance of the sensor 108 to determine concentration of the gas species to which the senor 108 is exposed. More particularly, the sensor 108 includes a metal oxide film 134, the electrical resistance of which is monitored by the sensing circuit 132 and controller 126. There is also a heater circuit 136 connecting between the controller 126 and a heater 138 of the sensor 108, which provides temperature control of the metal oxide film 134 for accurate gas species measurements.

The metal oxide film 134 and heater 138 are configured to detect hydrogen present or exposed to the metal oxide film 134 in a concentration range of 100 ppm (parts per million), to 10 ppt (parts per thousand), inclusive of endpoints. The sensor 108 is configured so a presence of hydrogen at 10 ppt yields a drop in electrical resistance across the sensor (across the metal oxide film 134) of at or below 10 kilo-Ohms.

Referring again to FIG. 3, a method of detecting perforated bowel during a surgical procedure includes detecting presence of a gas species, such as H₂ gas, indicative of a perforated bowel with a sensor, e.g. sensor 108, wherein the gas species reaches the sensor by diffusion from a main gas flow circuit, e.g. main gas flow circuit 104, of an gas recirculation system, e.g., gas recirculation system 102, maintaining pressure in a surgical cavity, e.g. surgical cavity 106. The method includes outputting an alert upon detection of the gas species concentration reaching a certain level, for example at, above, or near a threshold indicative of a perforated bowel, e.g. outputting audio and/or visual output through user interface 128.

The sensor is located in a channel, e.g. channel 110, branching off from the main gas flow circuit, which is a dead-end channel, wherein detecting includes detecting the gas species without bulk gas flow through the dead-end channel. Detecting can include sampling gas from the main gas flow circuit with the sensor, wherein sampling gas includes sampling the gas from a position in the main gas flow circuit downstream from a compressor, e.g. compressor 114, operatively connected to the gas recirculation system in the main gas flow circuit to move the flow of gas to and from the surgical cavity. Moving the flow of gas to and from the surgical cavity includes flowing gas to and from the surgical cavity through a gas sealed access port, e.g. gas sealed access port 122, connecting between the gas recirculation system and the surgical cavity, wherein the gas sealed access port and gas recirculation system form a sealed recirculation circuit with the gas sealed access port positioned in a surgical cavity.

A controller, e.g. controller 126, is operatively connected to the sensor determining if the gas species monitored in the gas flow from the surgical cavity is within a respective desired range and to initiate output to an output device to alert a user to take corrective action if the gas species is outside the respective desired range. The controller is operatively connected to a circuit, e.g. sensing circuit 132 in FIG. 3, of the sensor to monitor changes in electrical resistance of the sensor to determine concentration of the gas species to which the senor is exposed.

The method can include outputting a history of gas concentration detected by the sensor for a 1 to 60 second interval for recall upon gas levels of the gas species in excess of a predetermined threshold. The sensor includes a metal oxide film, e.g. film 134, and heater, e.g. heater 138, wherein detecting the gas species includes using the metal oxide film to detect hydrogen presence in a concentration range of 100 ppm (parts per million), to 10 ppt (parts per thousand), inclusive of endpoints.

The subject disclosure employs gas concentration measurements to alert the user to concentrations of bowel gases known to be emitted following bowel perforation. The measurement and associated alerts can aid surgeons in sensing a perforation and deciding on whether to extend surgery and anesthesia time to visually search for the perforation.

Insufflation is required for optical visualization during laparoscopic procedures. Standard insufflation delivers gas to the patient without any gas being returned to the gas recirculation system; however, insufflation using a gas sealed access port continuously returns air from the gas seal located adjacent to the patient while continuously drawing in gases from the inflated surgical site. Incorporating a gas sensor into the gas sealed recirculation circuit permits continuous automated gas monitoring without the need for additional equipment, additional surgical steps, or additional access ports.

By referencing a digital gas concentration record, the physician can potentially rule out the need for subsequent surgical exploration should a patient post-operatively develop symptoms associated with bowel perforation.

The methods and systems of the present disclosure, as described above and shown in the drawings, provide for monitoring gas species in a surgical site during endoscopic surgical procedures, e.g., to detect perforated bowels. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. A system for monitoring gas composition in a surgical cavity during an endoscopic surgical procedure, comprising:
a gas recirculation system including a main gas flow circuit for fluid communication between a surgical cavity and the gas recirculation system;
a flow channel branching off from the main gas flow; and
a sensor operatively associated with the flow channel for monitoring a gas species in the gas flow through the main gas flow circuit.

2. The system as recited in claim 1, wherein the channel branching off from the main gas flow circuit is a dead-end channel in the gas recirculation system.

3. The system as recited in claim 1, wherein the main gas flow circuit has a cross-sectional flow area with a main diameter, and wherein the sensor is spaced apart from the main gas flow circuit by a distance along the channel of ten or more main diameters distance so the sensor can detect the gas species reaching the sensor by diffusion through the channel from the main gas flow circuit.

4. The system as recited in claim 1, further comprising a compressor operatively connected to the gas recirculation system in the main gas flow circuit to move the flow of gas to and from the surgical cavity, wherein the channel connects to the main gas flow circuit at a position in the main gas flow circuit downstream from the compressor.

5. The system as recited in claim 4, wherein the main gas flow circuit includes an upstream portion for returning gas from the surgical cavity to the gas recirculation system, and a downstream portion for issuing gas to the surgical cavity, wherein the compressor separates between the upstream and downstream portions, and wherein the channel connects to the downstream portion.

6. The system as recited in claim 5, further comprising a gas sealed access port connected to the upstream portion and to the downstream portion for connecting the gas recirculation system to the surgical cavity.

7. The system as recited in claim 6, wherein the gas sealed access port, the upstream portion, the downstream portion, and the compressor are configured to form a continuous gas recirculation circuit with the gas sealed access port positioned in a surgical cavity.

8. The system as recited in claim 1, further comprising: a controller operatively connected to the sensor for determining if the gas species monitored in the gas flow from the surgical cavity is within a respective desired range and taking corrective action if the gas species is outside the respective desired range.

9. The system as recited in claim 8, wherein the controller is operatively connected to a circuit of the sensor to monitor changes in electrical resistance of the sensor to determine concentration of the gas species to which the senor is exposed.

10. The system as recited in claim 8, further comprising a user interface operatively connected to the controller, wherein the controller is configured to issue an alert to a user through the user interface upon detection of the gas species at a predetermined threshold.

11. The system as recited in claim 8, wherein the controller includes a memory, wherein the controller includes machine readable instructions configured to cause the controller to write a history of gas concentration detected by the sensor for a 1 to 60 second interval for recall upon gas levels in excess of a predetermined threshold.

12. The system as recited in claim 1, wherein the sensor includes a metal oxide film and heater, configured to detect hydrogen presence in a concentration range of 100 ppm (parts per million), to 10 ppt (parts per thousand), inclusive of endpoints.

13. The system as recited in claim 12, wherein the sensor is configured so a presence of hydrogen at 10 ppt yields a drop in electrical resistance across the sensor of at or below 10 kilo-Ohms.

14. A method of detecting perforated bowel during a surgical procedure, the method comprising:
detecting presence of a gas species indicative of a perforated bowel with a sensor, wherein the gas species reaches the sensor by diffusion from a main gas flow circuit of an gas recirculation in fluid communication with a surgical cavity; and
outputting an alert upon detection of the gas species at a threshold indicative of a perforated bowel.

15. The method as recited in claim 14, wherein the sensor is located in a channel branching off from the main gas flow circuit, wherein the channel is a dead-end channel, wherein detecting includes detecting the gas species without bulk gas flow through the dead-end channel.

16. The method as recited in claim 15, wherein the main gas flow circuit has a cross-sectional flow area with a main diameter, and wherein the sensor is spaced apart from the main gas flow circuit by a distance along the dead-end channel of ten or more main diameters.

17. The method as recited in claim 15, wherein detecting includes sampling gas from the main gas flow circuit with the sensor, wherein sampling gas includes sampling the gas from a position in the main gas flow circuit downstream from a compressor operatively connected to the gas recirculation system in the main gas flow circuit to move the flow of gas to and from the surgical cavity.

18. The method as recited in claim 17, wherein moving the flow of gas to and from the surgical cavity includes flowing gas to and from the surgical cavity through a gas sealed access port connecting between the gas recirculation system and the surgical cavity, wherein the gas sealed access port and gas recirculation system form a sealed recirculation circuit with the gas sealed access port positioned in a surgical cavity.

19. The method as recited in claim 14, wherein a controller operatively connected to the sensor determining if the gas species monitored in the gas flow from the surgical cavity is within a respective desired range and to initiate output to an output device to alert a user to take corrective action if the gas species is outside the respective desired range.

20. The method as recited in claim 19, wherein the controller is operatively connected to a circuit of the sensor to monitor changes in electrical resistance of the sensor to determine concentration of the gas species to which the senor is exposed.

21. The method as recited in claim 14, further comprising outputting a history of gas concentration detected by the sensor for a 1 to 60 second interval for recall upon gas levels of the gas species in excess of a predetermined threshold.

22. The method as recited in claim 14, wherein the sensor includes a metal oxide film and heater, and wherein detecting the gas species includes using the metal oxide film to detect hydrogen presence in a concentration range of 100 ppm (parts per million), to 10 ppt (parts per thousand), inclusive of endpoints.
